(19) United States
(12) Patent Application Publication
(10) Pub. No.: US 2005/0100977 A1
(43) Pub. Date: May 12, 2005

(54) Biochemical sensing device

(76) Inventors: Yuh-Shyong Yang, Hsinchu City (TW); Chung-Yu Wu, Hsinchu City (TW); Ude Lu, Taipei City (TW); Yu-Chuan Shih, Chiayi City (TW); Ben C.-P. Hu, Puli Township (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/772,365

(22) Filed: Feb. 6, 2004

(30) Foreign Application Priority Data
Nov. 7, 2003 (TW) 92131195

(51) Int. Cl. C12Q 1/60; C12Q 1/54; G01N 33/92; C12M 1/34

(52) U.S. Cl. 435/11; 435/14; 435/287.1; 436/71

(57) ABSTRACT

The invention is a biochemical sensing device, including a photodiode capable of sensing the light generated by the reaction made by a specific compound, a specific enzyme, and a luminol as well as converting the optical signal into a current signal. Also, there is a current/voltage converting circuit capable of converting the current signal into an analog voltage signal. In turn, the analog voltage signal can be converted into a digital voltage signal through an analog/digital converter. Finally, by using an electronic device, the digital voltage signal can be received and analyzed, and through the analysis, the amount of the specific compound can be measured. The device of the invention can provide a simple real-time medical assay that can be performed in massive amount. For this reason, the drawbacks of a conventional spectrum analysis instrument of being bulky and expensive can be improved.

### BACKGROUND OF THE INVENTION

1. Field of the Invention

The invention relates to a biochemical sensing device and, more particularly, to a biochemical sensing method and a sensing device that employs the light generated by a biochemical reaction and sensed by a photodiode to perform quantitative analysis on a specific compound.

2. Description of the Related Art

Advancements in modern scientific techniques such as biotechnology and electronic technology have propelled a global-wise revolution in medical techniques. With supports from all kinds of new techniques in medical examinations, a real-time personalized diagnosis has become significantly workable in daily life. Through real-time personalized diagnosis, a person can make a preliminary self-examination as well as save time for visiting a doctor. Therefore, it has become an important issue in medical industry to research and develop an assay device that is accurate, portable, cheap, and easy for use.

Among conventional medical examination instruments, the spectrum analysis tool, such as a spectrophotometer, is frequently used and therefore important for assaying. The spectrophotometer is to perform a qualitative or quantitative analysis on an object by applying a theory; that is, when light passes through the object, the object will absorb part of the light of wavelengths. Usually, the examination method for spectrum analysis is to dissolve an object to be tested into a specific solvent, and then the light of a light source will incident the solution. Meanwhile, some part of the light will be absorbed by the solute formed by the tested object, other part of the light will be reflected, and the rest part of the light will penetrate the solution. After that, the spectrum analysis tool can use a light sensing element to sense the penetrated light and find the absorbed spectrum curve and extinction coefficient of the given object according to the shape and strength of the absorbed spectrum curve. Then, the quantitative and qualitative analyses can be performed on the object. For instance, by employing the spectrum analysis tool, the glucose test, cholesterol test, uric acid test, and choline test can all be carried out.

However, a conventional spectrum analysis tool will utilize the photomultiplier tube (PMT) as a light sensing element. The photocathode of the PMT will emit electrons when stimulated by the photons. These electrons will impact the next dynode when accelerated by the electric field or magnetic field and then generate more electrons. If the electrons repeat the same accelerating impact process, the electron current will keep on amplifying. In the end, a high electric potential of anode will collect the amplified electron current. However, despite that the sensitivity of the PMT may be good, the need for high voltage (about 500 to 1000 volts), the size, and the price of the PMT limit its application in the spectrum analysis instruments. Therefore, if a person wants to use the spectrum analysis to perform health assay, a hospital seems to be the only place that can afford the assaying.

In view of the aforementioned problem, the invention provides a device capable of utilizing photodiode for performing biochemical sensing so as to improve the drawback of the conventional technique.

### SUMMARY OF THE INVENTION

The main and first object of the invention is to provide a biochemical sensing device, which utilizes a sensing element fabricated by the complementary metal oxide semiconductor (CMOS) process to sense the luminescence light, fluorescence light, and ultraviolet light generated by biochemical reaction, thereby reducing the cost and volume of the instrument for assaying.

The second object of the invention is to provide a biochemical sensing device for supporting real time assays in massive amount.

The third object of the invention is to provide a biochemical sensing device capable of simplifying a biomedical assay into a procedure that can be operated by a common user.

The biochemical sensing device of the invention includes a bearing body that bears a specific compound, a luminol, and a luminescence assay reagent formed by mixing a first enzyme and a second enzyme. The chemiluminescence generated by the luminescence assay reagent will be sensed by a sensing element fabricated by a semiconductor process. In turn, the sensing element will convert the sensed light into a current signal. Also, there is a current/voltage converting circuit capable of converting a current signal into an analog voltage signal. The analog voltage signal, on the other hand, can be converted into a digital voltage signal through an analog/digital converter. Finally, there is an electronic device to receive the digital voltage signal and then analyze, store, and transmit the signal.

The objects and technical contents of the invention will be better understood through the description of the following embodiments with reference to the drawings.

### BRIEF DESCRIPTION OF THE DRAWINGS

FIG. 1 is a schematic diagram showing the biochemical sensing process flow of the invention.

FIG. 2 is a block schematic diagram showing the biochemical sensing device of the invention.

FIG. 3 is a schematic diagram showing the CMOS sensing chip circuit of the invention.

FIG. 4 is a schematic diagram showing the biochemical sensing device of the invention.

FIG. 5 is a schematic diagram showing the measured curve of horseradish peroxidase according to the invention.

FIG. 6 is a schematic diagram showing the measured curve of hydrogen peroxide (H₂O₂) according to the invention.

FIG. 7 is a schematic diagram showing the measured curve of glucose according to the invention.

### DETAILED DESCRIPTION OF THE PREFERRED EMBODIMENTS

The invention is a biochemical sensing device. By employing the device, the cost and volume of an assay instrument can be reduced as well as a real-time and do-it-yourself assay can be performed by a common user so as to reduce time and cost for visiting a doctor.

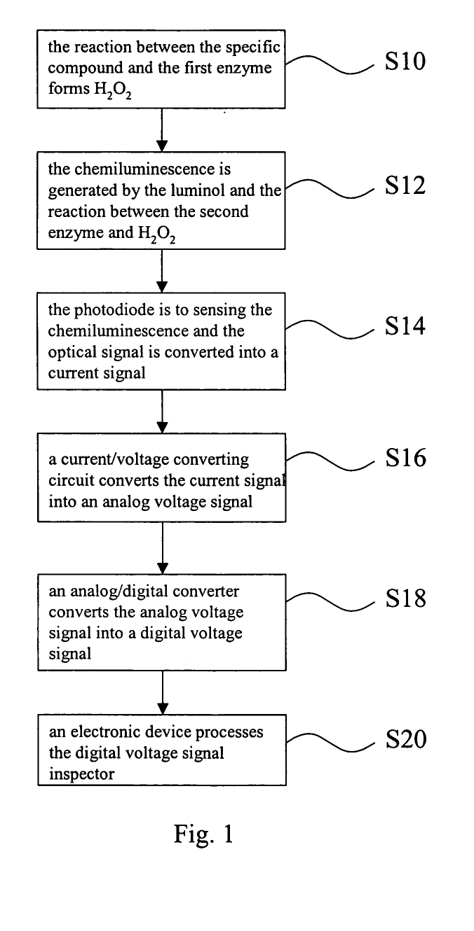

FIG. 1 is a schematic diagram showing the biochemical sensing process flow of the invention. Referring to FIG. 1, first in the step S10, a solution to be tested containing specific compound will be mixed with a solution containing a first enzyme. Then, the mixed solution produces a chemical reaction and generates H₂O₂. The specific compound can be a human body compound such as glucose, cholesterol, uric acid, lactate, phospholipids, and triglycerides. As to the first enzyme, it has to be selected appropriately according to the type of specific compound that exists. For example, the type of first enzyme can be glucose oxidase, cholesterol oxidase, uricase, lactate oxidase, phospholipase, and lipase. After the step S10, the luminol reagent and the peroxidase in the step S12 will generate chemiluminescence when they are producing a reaction with H₂O₂ generated in the step S10. Through the chemiluminescence reaction, the luminescence light, fluorescence light, or ultraviolet light can be generated. In addition, the luminol reagent used in the step S12 is selected from the chemiluminescence reagent, for instance, luminol, 2-methyl indole, isoluminol, dioxetane, acridinium ester, lucigenin, AMPPD, CDP-Star, and CSPD. Next, after the step S12 is finished, the step S14 will be executed, which is to employ the sensing element fabricated by at least one semiconductor process, such as a photodiode, to sense the light generated in the step S12. The photodiode will also convert the sensed optical signal into a current signal. Then, in the step S16, the current signal will be converted into an analog voltage signal through a current/voltage converting circuit. The current/voltage converting circuit can also amplify the current signal by the design of current mirror, whereas the current signal can be converted into an analog voltage signal by a resistor or a capacitor. After the step S16, the procedure moves into the step S18, in which the analog voltage signal can be converted into a digital voltage signal through an analog/digital converter. Finally, in the step S20, the digital voltage signal can be analyzed, stored, or transmitted by means of an electronic device, for instance, a portable displayer, a personal computer, or a workstation. Therefore, the information about how much specific compound contained in the solution can be obtained.

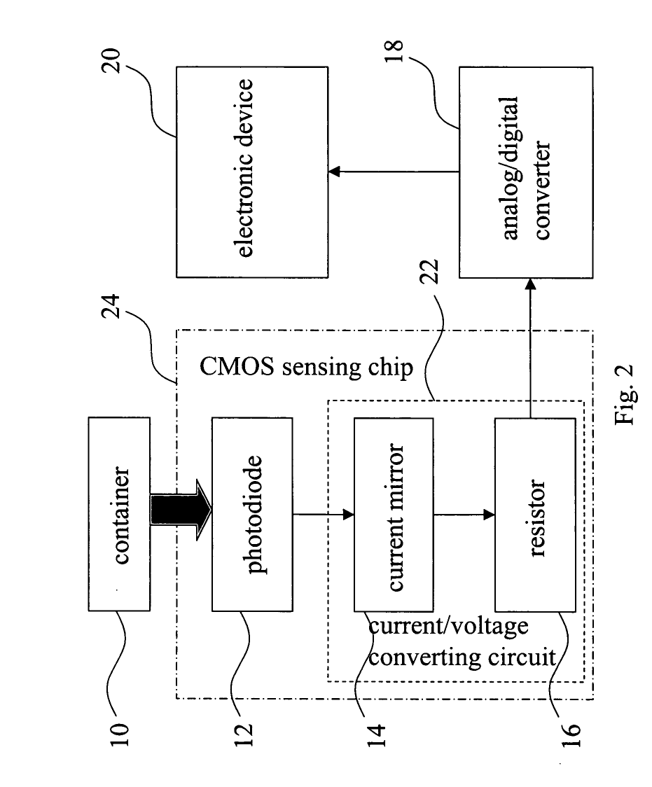

Furthermore, FIG. 2 is a block schematic diagram showing the biochemical sensing device of the invention. Referring to FIG. 2, a container 10 contains biochemical luminescence assay reagent. The reagent contains a specific compound, a first enzyme, a luminol, and a peroxidase. The specific compound and the first enzyme will produce a reaction and generate H₂O₂, whereas the H₂O₂, the luminol, and the peroxidase will produce another reaction and generate luminescence light, fluorescence light, or ultraviolet light. Then, the light generated by the chemiluminescence will be sensed by a photodiode 12, and the photodiode 12 will also convert the sensed optical signal into a current signal. However, in order to prevent the current signal from being too weak to be recognized, a current mirror 14 will amplify the current signal. After that, a resistor 16 will convert the amplified current signal into an analog voltage signal for signal processing. Finally, an analog/digital converter 18 will convert the analog voltage signal into a digital voltage signal, and an electronic device 20 will receive the digital voltage signal for further analysis, storage, or transmission in order to obtain information about the concentration of specific compound contained in the reagent.

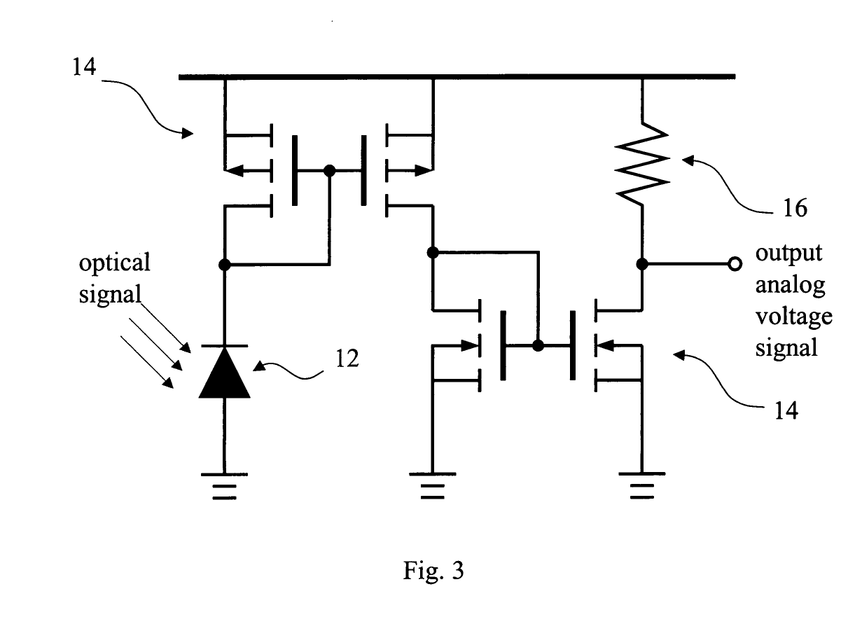

Moreover, referring to FIG. 2, the container 10 can be a bearing body such as a quartz glass tube or a testing slide ( [symbol] ?), in which the specific compound it contains can be a body compound, such as glucose, cholesterol, uric acid, lactate, phospholipids, and triglycerides. As to the first enzyme, it has to be selected appropriately according to the type of specific compound that exists. The luminol reagent can be selected from the chemiluminescence reagent, such as luminol, 2-methyl indole, isoluminol, dioxetane, acridinium ester, lucigenin, AMPPD, CDP-Star, and CSPD. Also, the current mirror 14 and the resistor 16 can be integrated with a current/voltage converting circuit 22, and the resistor 16 can be replaced by a capacitor. Through the current/voltage converting circuit 22, the current signal can be converted into the analog voltage signal so as to be outputted to the analog/digital converter. Moreover, the photodiode 12, the current mirror 14, and the resistor 16 can be integrated on a CMOS sensing chip 24, enabling the CMOS sensing chip 24 to have functions of sensing the chemiluminescence as well as converting the optical signal into an analog voltage signal for outputting, as shown in FIG. 3.

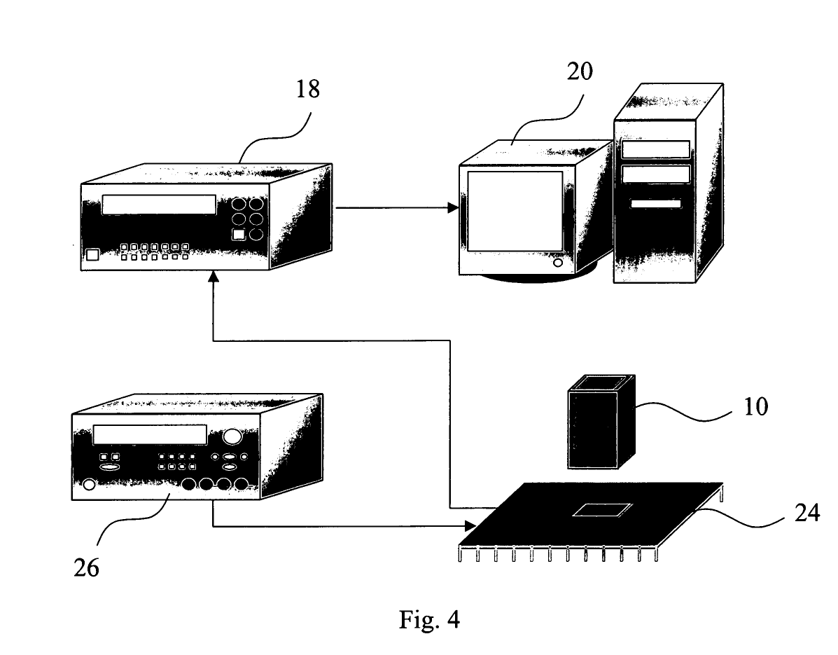

Also, FIG. 4 is a schematic diagram showing the biochemical sensing device of the invention. As shown in FIG. 4, the CMOS sensing chip 24 includes the aforementioned photodiode and the current/voltage converting circuit. Besides, a container 10 is placed on the CMOS sensing chip 24 and contains biochemical luminescence assay reagent. The reagent will produce chemiluminescent reaction and generate luminescence light, fluorescence light, and ultraviolet light. Then, the generated light can be sensed by the CMOS sensing chip 24, and the CMOS sensing chip 24 will convert the optical signal into an analog voltage signal and output it to an analog/digital converter 18. Then, the analog/digital converter 18 will convert the analog voltage signal into a digital voltage signal so as to be outputted to an electronic device 20, which in turn will analyze, store, or transmit the digital voltage signal. Finally, there is a voltage supplier 26 that can provide stable voltage source to the CMOS sensing chip 24.

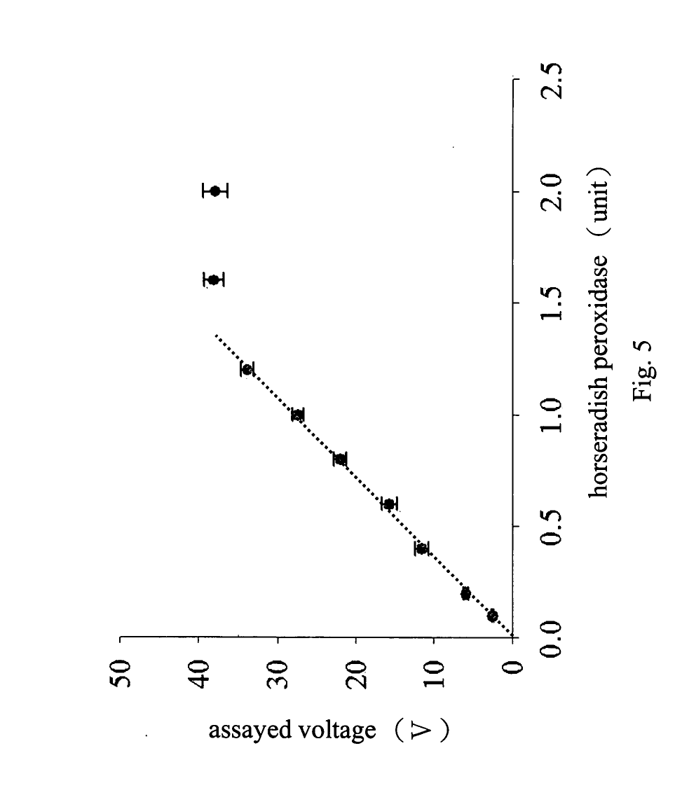

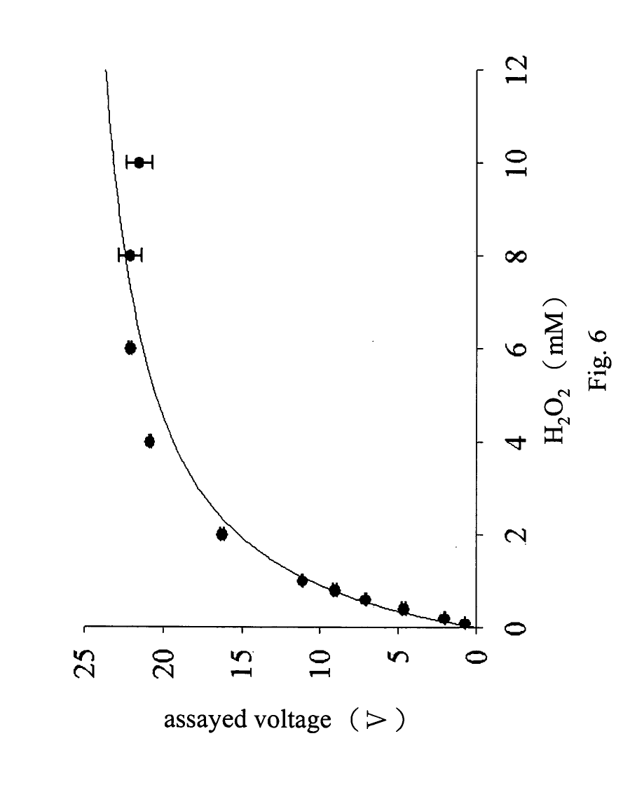

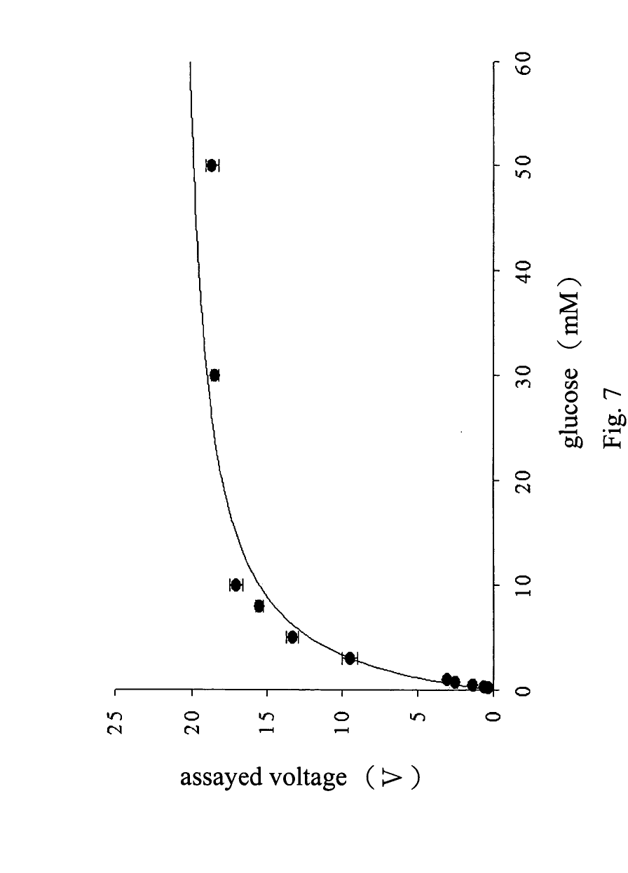

Furthermore, the invention also performs quantitative analysis on the glucose to verify the feasibility of the invention. First, the glucose and the glucose oxidase will produce a reaction and generate H₂O₂, wherein the chemical reaction is: 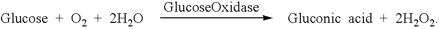
Next, the H₂O₂ and the horseradish peroxidase will produce a reaction and generate the chemiluminescence, wherein the chmical reaction is: 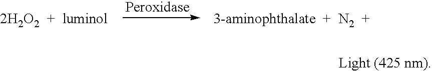
Finally, the biochemical sensing device of the invention will sense the light generated by the chemiluminescent reaction for measuring the quantity of glucose. Also, FIG. 5, FIG. 6, and FIG. 7 show the measured curves of horseradish peroxidase, H₂O₂, and glucose, respectively according to the invention. As shown in FIG. 5, when the horseradish peroxidase is under 1 unit, the activity of horseradish peroxidase will be directly proportional to the voltage measured by the biochemical sensing device of the invention. One unit of horseradish peroxidase means that 1 mg of purpurogallin can be catalyzed from pyrogallol in 20 seconds at PH 6.0 at 20° C. FIG. 6 shows relationship between the concentration of H₂O₂ and the measured voltage. Finally, it can be understood from the measured curve of glucose shown in FIG. 7 that if the concentration of glucose is under 5 mM, the measured voltage will have an obvious change when the concentration of glucose has a slight change. Therefore, the invention actually can perform a precise quantitative analysis on the glucose by employing the glucose curve.

In conclusion, the invention employs a CMOS sensing chip fabricated by a sophisticated CMOS process to sense the light generated by the chemiluminescent reaction. Then, electronic devices such as an analog/digital converter and a portable displayer are utilized for processing the signals sensed by the CMOS sensing chip. In addition, because the CMOS sensing chip has the advantages of being small in volume, low in cost and suitable for mass production, and because the analog/digital converter can be integrated with the CMOS sensing chip or the electronic device, the invention can provide a low-cost, precise and convenient real-time assay so that a user is able to perform a self health-examination without visiting a doctor. Consequently, the time for visiting a doctor can be saved, and the cost of medical care can then be reduced. Above all, a personalized health examination can be performed at any time a user desires to do so.

The embodiments above are only intended to illustrate the invention; they do not, however, to limit the invention to the specific embodiments. Accordingly, various modifications and changes may be made without departing from the spirit and scope of the invention as described in the following claims.

1. A biochemical sensing device, including:
a bearing body, which bears a reagent thereon, and the reagent contains a specific compound, a first enzyme, a second enzyme, and a luminol, in which the specific compound and the first enzyme will produce a reaction and then generate H₂O₂, whereas the H₂O₂, the second enzyme, and the luminol will produce a chemiluminescent reaction;
a sensing element, fabricated by a semiconductor process for sensing the light generated by the chemiluminescent reaction as well as for converting the sensed optical signal into a current signal;
a current/voltage converting circuit, capable of converting the current signal into a voltage signal, and
an electronic device, which can receive and process the voltage signal so as to perform a quantitative analysis on the specific compound.

2. The biochemical sensing device as claimed in claim 1, wherein the specific compound is selected from the following human body compound: glucose, cholesterol, uric acid, lactate, phospholipids, and triglycerides.

3. The biochemical sensing device as claimed in claim 1, wherein the luminol reagent can be selected from the following chemiluminescent reagent: luminol, 2-methyl indole, isoluminol, dioxetane, acridinium ester, lucigenin, AMPPD, CDP-Star, and CSPD.

4. The biochemical sensing device as claimed in claim 1, wherein the first enzyme is selected appropriately according to the type of specific compound that exists.

5. The biochemical sensing device as claimed in claim 1, wherein the second enzyme is peroxidase.

6. The biochemical sensing device as claimed in claim 1, wherein the sensing element can be a photodiode.

7. The biochemical sensing device as claimed in claim 1, wherein the sensing element is designed for sensing the luminescence light, fluorescence light, and ultraviolet light, or any combination of the three.

8. The biochemical sensing device as claimed in claim 1, wherein the current/voltage converting circuit can include at least one current mirror so as to amplify the current signal.

9. The biochemical sensing device as claimed in claim 1, wherein the current/voltage converting circuit can include at least one resistor so as to convert the current signal into an analog voltage signal.

10. The biochemical sensing device as claimed in claim 1, wherein the current/voltage converting circuit can include at least one capacitor so as to convert the current signal into an analog voltage signal.

11. The biochemical sensing device as claimed in claim 9, wherein the current/voltage converting circuit can include an analog/digital converter so as to convert the analog voltage signal into the digital voltage signal.

12. The biochemical sensing device as claimed in claim 10, wherein the current/voltage converting circuit can include an analog/digital converter so as to convert the analog voltage signal into the digital voltage signal.

13. The biochemical sensing device as claimed in claim 9, wherein the electronic device can include an analog/digital converter so as to convert the analog voltage signal into the digital voltage signal.

14. The biochemical sensing device as claimed in claim 10, wherein the electronic device can include an analog/digital converter so as to convert the analog voltage signal into the digital voltage signal.

15. The biochemical sensing device as claimed in claim 1, wherein the processing method used by the electronic device to process the voltage signal is selected from transmission, storage, and analysis or any combination of the three.
